(19) United States
(12) Patent Application Publication
(10) Pub. No.: US 2005/0267488 A1
(43) Pub. Date: Dec. 1, 2005

(54) Apparatus and method for using an ultrasonic medical device to treat urolithiasis

(75) Inventors: Bradley A. Hare, Chelmsford, MA (US); Robert A. Rabiner, North Reading, MA (US); Rebecca I. Marciante, North Reading, MA (US); Mark J. Varady, Andover, MA (US)

Correspondence Address: FISH & RICHARDSON PC, P.O. BOX 1022, MINNEAPOLIS, MN, 55440-1022, US

(73) Assignee: OmniSonics Medical Technologies, Inc.

(21) Appl. No.: 10/844,861

(22) Filed: May 13, 2004

(51) Int. Cl. A61B 17/24

(52) U.S. Cl. 606/113

(57) ABSTRACT

An apparatus and a method for an ultrasonic medical device to treat urolithiasis and ablate a stone. The ultrasonic medical device comprises an ultrasonic probe having a wire body with a proximal end, a distal end and a longitudinal axis therebetween and a plurality of tines extending from the distal end of the wire body. The ultrasonic medical device includes a sheath capable of surrounding the wire body and the plurality of tines. The ultrasonic probe is inserted into the sheath and the ultrasonic probe is moved until the plurality of tines surround at least a portion of an outer surface of the stone. An ultrasonic energy source engaged to the ultrasonic probe supplies an ultrasonic energy to the ultrasonic probe to produce a transverse ultrasonic vibration along at least a portion of the ultrasonic probe to ablate the stone.

### RELATED APPLICATIONS

None.

### FIELD OF THE INVENTION

The present invention relates to medical devices, and more particularly to an apparatus and a method for an ultrasonic medical device to treat urolithiasis.

### BACKGROUND OF THE INVENTION

Urolithiasis is a condition in which crystals, or mineral deposits, in the urine combine to form stones. The stones, also referred to as calculi or uroliths, can be found anywhere in the urinary tract or bladder and result in the obstruction of the urethra. Urolithiasis is caused by an imbalance of calcium and phosphorus in the diet. The stones cause irritation, discomfort, can result in a secondary infection and in some cases has led to death. The types of stones for the urolithiasis condition include kidney stones, cystine kidney stones, struvite stones, urate stones, calcium oxalate stones, gall stones, urinary stones, bladder stones, cystine stones, xanthine stones, calcium phosphate stones, endemic bladder stones, oxylate stones, renal stones, uric acid stones and uric acid plus calcium stones, among others.

Development of the urolithiasis condition can depend on a genetic predisposition. The inheritance of some renal stone diseases is common, with some reports indicating that as many as about 70% of children with idiopathetic hypercalciuria have a family history of stones. Cystinuria, an autosomal recessive defect of amino acid transport, can lead to cystine kidney stones. Oxylate stones can be produced as a result of the rare inherited renal tubular defect of glycinuria as well as the autosomal disorder of primary hyperoxalurai. Other inherited disorders in purine metabolism lead to uric acid stones. Xanthine stones are produced from the autosomal recessive disorder of xanthinuria.

Reports have indicated that a number of dietary items contribute to renal stone production. Calcium oxalate stone production may result from a high oxalate intake. Stones containing uric acid and uric acid plus calcium components may result from excessive purine intake. The ketogenic diet, a diet that is prescribed to reduce seizures, increases the risk of uric acid stone and calcium stone formation in children. High protein diets in which protein is derived from animal sources, glucose or sucrose increases urinary calcium and may lead to stone formation.

Excessive intake of vitamin A and vitamin D can contribute to calcium urolithiasis. A low fluid intake promotes concentrated urine and increases the risk of stone formation. Drug intake contributes to stone formation. Anticancer agents increase the filtered load of uric acid increasing the risk of uric acid stone formation. Glucocorticoids increase the filtered load of calcium, leading to calcium stone formation. Allopurinol increases the filtered load of xanthine in patients with tumor lysis to produce xanthinuria, leading to the formation of xanthine stones. Other diseases or the medications used to treat diseases increase stone formation risk.

Urolithiasis is a condition that affects millions of people worldwide and is a common condition for several animals including dogs, cats, lambs, calves, cows and horses. While there are not an abundant amount of studies of urolithiasis, some trends and commonalities can be seen. Studies have shown that the frequency of urolithiasis is about 4 times greater in men than in women. Studies of the younger population have shown that boys are more apt to have urolithiasis than girls, with the frequency ratio about 3 to about 2. The peak presentation of urolithiasis for adults is the middle age years. For children in the United States, the incidence of urolithiasis varies between about 1 case per 1000 and 1 case per 7600 hospital admissions. Regional trends in the United States have been seen, with the Southeast United States having a higher frequency of kidney stone formation than other United States regions. Suggested factors for the regional trend range from climate, diet, genetics, state of hydration and bacterial colonization. The frequency of urolithiasis is higher in developing countries, where dietary protein is derived mostly from cereal grains or plant sources as opposed to meats.

The presence of stones in various organs of the body including the bladder and the urethra can be painful. The prior art has not addressed the problem of effectively ablating stones within the body in a safe and efficient manner. Prior art devices to remove stones are inadequate and subject patients with the stones to unnecessary health risks. Prior art devices utilize both invasive and non-invasive techniques for the removal of the stone from the organ in the body.

U.S. Pat. No. 4,696,299 to Shene et al. discloses an apparatus for the non-invasive disintegration of kidney stones and the like. The Shene et al. device comprises an ellipsoidal reflector that is positioned against the body, whereby a series of sparks are discharged across a spark gap located at the first focal point of the ellipsoid. The sparks generate a series of shock waves that travel through water in the reflector and through the body to impinge on the stone and disintegrate the stone. The Shene et al. device does not effectively focus the shock waves directly on the stone and utilizes an unreliable method of ensuring that the shock waves are targeted at the stone. Disintegration of the stone with the Shene et al. device requires several attempts of positioning the reflector against the body and generating sparks and is a time consuming process that does not reduce the stones to a size that can be easily discharged from the body.

U.S. Pat. No. 6,551,327 to Dhindsa discloses an endoscopic stone extraction device with improved basket. The Dhindsa device includes a handle that supports a sheath and a filament that supports a stone extraction basket. The basket comprises a stone retention region comprising small openings that retain stones smaller than two millimeters in diameter. The Dhindsa device is optimized for the extraction of stone fragments smaller than two millimeters in diameter, and would subject the patient to pain as the stone is removed from the organ. The Dhindsa device does not provide a way of fragmenting the stone in order to reduce the stone to a size that can be easily removed through the body in conventional ways.

U.S. Pat. No. 4,474,180 to Angulo discloses an apparatus for disintegrating kidney stones comprising a catheter, a waveguide, an ultrasonic transducer and a vibrational output. The device is inserted into an organ of the body such as the urethra until the distal end of the waveguide contacts the kidney stone. The ultrasonic transducer imparts longitudinal vibrations that are transmitted to the waveguide to create vibrational energy that breaks up the stones. The Angulo device has a large diameter that subjects the patient to pain when the Angulo device is inserted into small organs of the body. Since the Angulo device utilizes a technique of shattering the stones, the stone fragments are scattered within the organ after the vibrational energy is imparted, making it difficult to collect all of the fragments for removal or for a subsequent treatment.

The prior art devices do not solve the problem of effectively ablating stones within the body in a safe and efficient manner. The prior art devices do not effectively capture and ablate the stone to a size such that the particulate can be discharged from the body in conventional ways. The prior art devices subject patients to pain and lack desired safety. Therefore, there remains a need in the art for an ultrasonic medical device to treat urolithiasis that captures the stone, contains the stone, effectively ablates the stone to a particulate that can be easily discharged from the body in conventional ways and does not subject the patient to unnecessary pain.

### SUMMARY OF THE INVENTION

The present invention provides an apparatus and a method for an ultrasonic medical device to treat urolithiasis by removing a stone in an organ of a body. The ultrasonic medical device includes a wire body having a proximal end, a distal end and a longitudinal axis therebetween and a plurality of tines extend from the distal end of the wire body to engage the stone. The ultrasonic medical device also includes a sheath capable of surrounding the wire body and the plurality of tines.

The present invention is an ultrasonic probe for ablation of at least one stone in an organ of a body. The ultrasonic probe comprises a wire body having a proximal end, a distal end and a longitudinal axis therebetween and a plurality of tines engaging the wire body. An ultrasonic energy source engaged to the ultrasonic probe supplies an ultrasonic energy to the ultrasonic probe, producing a transverse ultrasonic vibration along at least a portion of the ultrasonic probe to ablate the stone.

The present invention is a method of ablating a stone in an organ of a body comprising: inserting an ultrasonic probe into a sheath, the ultrasonic probe having a wire body and a plurality of tines extending from a distal end of the wire body; moving the plurality of tines from a collapsed position to an expanded position by advancing the plurality of tines beyond a distal end of the sheath; moving the ultrasonic probe until the plurality of tines surround at least a portion of an outer surface of the stone; compressing a portion of the plurality of tines to engage the stone; and activating an ultrasonic energy source to provide an ultrasonic energy to the ultrasonic probe to ablate the stone.

The present invention is a method of reducing a size of at least one stone in an organ of a body. An ultrasonic probe having a wire body with a plurality of tines engaging the wire body is inserted into a biocompatible material member. The plurality of tines are moved from a collapsed position to an expanded position by advancing the plurality of tines beyond a distal end of the biocompatible material member. The ultrasonic probe is moved until the plurality of tines surround at least a portion of an outer surface of at least one stone. An ultrasonic energy source is activated to produce a transverse ultrasonic vibration along the ultrasonic probe to reduce the size of the stone.

The present invention provides an apparatus and a method for treating urolithiasis. An ultrasonic probe having a wire body with a plurality of tines extending from a distal end of the wire body is moved so the plurality of tines surround at least a portion of an outer surface of the stone. An ultrasonic energy from an ultrasonic energy source ablates the stone. The present invention provides an ultrasonic medical device for treating urolithiasis that is simple, user-friendly, time efficient, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a side plan view of an ultrasonic medical device of the present invention capable of operating in a transverse mode having an ultrasonic probe with a wire body and a plurality of tines extending from a distal end of the wire body, the plurality of tines connected at a distal end, and a sheath surrounding a portion of the ultrasonic probe.

FIG. 2 is a fragmentary side plan view of an ultrasonic probe of the present invention having a plurality of tines connected at a distal end and in a collapsed position.

FIG. 3 is a fragmentary side plan view of an ultrasonic probe of the present invention having a plurality of tines connected at a distal end and in an expanded position.

FIG. 4 is a perspective end view of a preferred embodiment of a plurality of tines of FIG. 3 having four tines.

FIG. 5 is a fragmentary side plan view of an ultrasonic probe of the present invention in an organ of a body with a plurality of tines connected at a distal end surrounding a stone.

FIG. 6A is a view of an ultrasonic probe of the present invention inserted through a urethra of a patient and moved inside of a kidney to ablate a stone.

FIG. 6B is a view of an ultrasonic probe of the present invention inserted through a patient's back and moved inside of the kidney to ablate a stone.

FIG. 6C is a fragmentary side plan view of an ultrasonic probe of the present invention in an organ of the body with a plurality of tines connected at a distal end surrounding a stone and a sheath compressing a portion of the plurality of tines.

FIG. 7 is an exploded side plan view of a plurality of tines of an ultrasonic probe of the present invention showing a plurality of transverse nodes and a plurality of transverse anti-nodes along a plurality of tines.

FIG. 8 is a fragmentary side plan view of an ultrasonic probe of the present invention in an organ of a body showing a stone has been ablated, with a sheath compressing a portion of a plurality of tines that are connected at a distal end.

FIG. 9 is a fragmentary side plan view of an alternative embodiment of an ultrasonic probe of the present invention having a plurality of wire body segments and a plurality of tines connected at a distal end.

FIG. 10A is a perspective end view of an alternative embodiment of the present invention showing a plurality of tines having five tines.

FIG. 10B is a perspective end view of an alternative embodiment of the present invention showing a plurality of tines having three tines.

FIG. 10C is a perspective end view of an alternative embodiment of the present invention showing a plurality of tines having two tines.

FIG. 11 is a side plan view of an alternative embodiment of an ultrasonic medical device of the present invention capable of operating in a transverse mode having an ultrasonic probe with a wire body and a plurality of tines extending from a distal end of the wire body, the plurality of tines not connected at a distal end and a sheath surrounding a portion of the ultrasonic probe.

FIG. 12 is a fragmentary side plan view of an alternative embodiment of an ultrasonic probe of the present invention having a plurality of tines not connected at a distal end and in a collapsed position.

FIG. 13 is a fragmentary side plan view of an alternative embodiment of an ultrasonic probe of the present invention having a plurality of tines not connected at a distal end and in an expanded position.

FIG. 14 is a perspective end view of an alternative embodiment of a plurality of tines not connected having four tines.

FIG. 15 is a fragmentary side plan view of an alternative embodiment of an ultrasonic probe of the present invention in an organ of a body with a plurality of tines not connected at a distal end surrounding a stone.

FIG. 16 is a fragmentary side plan view of an alternative embodiment of an ultrasonic probe of the present invention in an organ of the body with a plurality of tines not connected at a distal end surrounding a stone and a sheath compressing a portion of the plurality of tines.

FIG. 17 is a side plan view of a plurality of tines of an alternative embodiment of an ultrasonic probe of the present invention showing a plurality of transverse nodes and a plurality of transverse anti-nodes along the plurality of tines.

FIG. 18 is a fragmentary side plan of an alternative embodiment of an ultrasonic probe of the present invention in an organ of a body showing a stone has been ablated, with a sheath compressing a portion of a plurality of tines that are not connected at a distal end.

FIG. 19 is a side plan view of an alternative embodiment of an ultrasonic probe of the present invention having a plurality of wire body segments, a plurality of tines connected at one location and a plurality of tines not connected at a distal end of the ultrasonic probe.

FIG. 20A is a perspective end view of an alternative embodiment of the present invention having a plurality of tines with five tines.

FIG. 20B is a perspective end view of an alternative embodiment of the present invention having a plurality of tines with three tines.

FIG. 20C is a perspective end view of an alternative embodiment of the present invention having a plurality of tines with two tines.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for the removal of a stone in an organ of a body using an ultrasonic medical device comprising an ultrasonic probe with a wire body and a plurality of tines extending from a distal end of the wire body and a sheath surrounding a portion of the ultrasonic probe. The plurality of tines surround at least a portion of an outer surface of the stone and increase a surface area of the ultrasonic probe in communication with the stone. The plurality of tines is movable between a collapsed position and an expanded position. In a preferred embodiment of the present invention, the plurality of tines are connected at a distal end of the ultrasonic probe. An ultrasonic energy source engaged to the ultrasonic probe supplies an ultrasonic energy to the ultrasonic probe, producing a transverse ultrasonic vibration along at least a portion of the ultrasonic probe including the plurality of tines to ablate the stone.

The following terms and definitions are used herein:

“Ablate” as used herein refers to removing, clearing, destroying or taking away a biological material. “Ablation” as used herein refers to a removal, clearance, destruction, or taking away of the biological material.

“Anti-node” as used herein refers to a region of a maximum energy emitted by an ultrasonic probe at or proximal to a specific location along a longitudinal axis of the ultrasonic probe.

“Node” as used herein refers to a region of a minimum energy emitted by an ultrasonic probe at or proximal to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe).

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Biological material” as used herein refers to a collection of a matter including, but not limited to, a group of similar cells, occlusions, plaque, intravascular blood clots or thrombus, fibrin, calcified plaque, calcium deposits, occlusional deposits, atherosclerotic plaque, fatty deposits, adipose tissues, atherosclerotic cholesterol buildup, fibrous material buildup, arterial stenoses, minerals, high water content tissues, platelets, cellular debris, wastes and other occlusive materials.

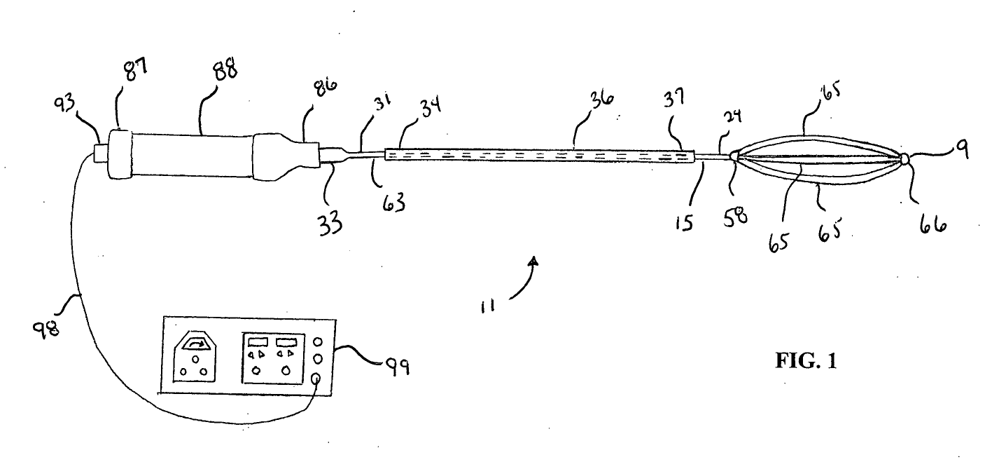

An ultrasonic medical device of the present invention is generally shown at 11 in FIG. 1. The ultrasonic medical device 11 includes an ultrasonic probe 15 which is coupled to an ultrasonic energy source or generator 99 for the production of an ultrasonic energy. A handle 88, comprising a proximal end 87 and a distal end 86, surrounds a transducer within the handle 88. The transducer, having a first end engaging the ultrasonic energy source 99 and a second end coupled to a proximal end 31 of the ultrasonic probe 15, transmits the ultrasonic energy to the ultrasonic probe 15. A connector 93 and a connecting wire 98 engage the ultrasonic energy source 99 to the transducer. The ultrasonic probe 15 includes a wire body 63 having the proximal end 31 and a distal end 24 of the wire body 63. A plurality of tines 65 extend from the distal end 24 of the wire body 63 and surround at least a portion of a stone in an organ of a body. The plurality of tines 65 form a basket-like structure. In a preferred embodiment of the present invention, a proximal end 58 of the plurality of tines 65 at a proximal end 58 of the basket 67 is welded to the wire body 63 and a distal end 66 of the plurality of tines 65 are connected, ending in a probe tip 9. A sheath 36 having a proximal end 34 and a distal end 37 surrounds at least a portion of a longitudinal axis of the ultrasonic probe 15. A coupling 33 that engages the proximal end 31 of the ultrasonic probe 15 to the transducer within the handle 88 is illustrated generally in FIG. 1. In a preferred embodiment of the present invention, the coupling is a quick attachment-detachment system. An ultrasonic probe device with a quick attachment-detachment system is described in the Assignee's U.S. Pat. No. 6,695,782 and co-pending patent applications U.S. Ser. No. 10/268,487 and U.S. Ser. No. 10/268,843, and the entirety of all these patents and patent applications are hereby incorporated herein by reference.

The sheath 36 is a thin, flexible, hollow tube that is small enough to be threaded through an organ of the body or the vasculatures that lead to the organ of the body. Patients generally do not feel the movement of the sheath 36 through the body. Once in place, the sheath 36 allows a number of tests or other treatment procedures to be performed. An ultrasonic medical device for ablation and a sheath for use therewith is disclosed in Assignee's U.S. Pat. No. 6,524,251, and the entirety of this patent is hereby incorporated herein by reference. Those skilled in the art will recognize that many sheaths known in the art can be used with the present invention and still be within the spirit and scope of the present invention.

In an embodiment of the present invention, the sheath 36 is comprised of polytetrafluoroethylene (PTFE). In another embodiment of the present invention, the sheath 36 is comprised of nylon. In another embodiment of the present invention, the sheath 36 is comprised of a polymer material. Those skilled in the art will recognize the sheath can be made of many materials known in the art and be within the spirit and scope of the present invention.

In another embodiment of the present invention, a biocompatible material member surrounds a portion of the longitudinal axis of the ultrasonic probe 15. The biocompatible material member includes, but is not limited to, a catheter, a balloon, a sheath and similar members. Those skilled in the art will recognize that other biocompatible material members known in the art would be within the spirit and scope of the present invention.

The ultrasonic probe 15 has a stiffness that gives the ultrasonic probe 15 a flexibility so it can be articulated in an organ of a body and through the vasculatures that may lead to the organ. The flexibility of the ultrasonic probe 15 allows the ultrasonic probe 15 to be deflected, flexed and bent through the vasculature to reach areas in the vasculature that would otherwise be difficult to reach. In an embodiment of the present invention, the diameter of the wire body 63 of ultrasonic probe 15 decreases from a first defined interval to a second defined interval along a longitudinal axis of the wire body. In another embodiment of the present invention, the diameter of the ultrasonic probe 15 decreases at greater than two defined intervals. The diameter of the ultrasonic probe 15 decreases from a first defined interval to a second defined interval across a transition. In an embodiment of the present invention, the transitions of the ultrasonic probe 15 are tapered to gradually change the diameter from the proximal end 31 of the ultrasonic probe 15 to the distal end 24 of the wire body 63 along the longitudinal axis of the ultrasonic probe 15. In another embodiment of the present invention, the transitions of the ultrasonic probe 15 are stepwise to change the diameter from the proximal end 31 to the distal end 24 along the longitudinal axis of the ultrasonic probe 15. Those skilled in the art will recognize that there can be any number of defined intervals and transitions, and that the transitions can be of any shape known in the art and be within the spirit and scope of the present invention.

The probe tip 9 can be any shape including, but not limited to, rounded, bent, a ball or larger shapes. In a preferred embodiment of the present invention, the probe tip 9 is smooth to prevent damage to the vasculature. In one embodiment of the present invention, the ultrasonic energy source 99 is a physical part of the ultrasonic medical device 11. In another embodiment of the present invention, the ultrasonic energy source 99 is not a physical part of the ultrasonic medical device 11.

In a preferred embodiment of the present invention, a cross section of the ultrasonic probe 15 is approximately circular. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

The ultrasonic probe 15 is inserted into the vasculature and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

In a preferred embodiment of the present invention, the ultrasonic probe 15 comprises titanium or a titanium alloy. Titanium is a strong, flexible, low density, low radiopacity and easily fabricated metal that is used as a structural material. Titanium and its alloys have excellent corrosion resistance in many environments and have good elevated temperature properties. In a preferred embodiment of the present invention, the ultrasonic probe 15 comprises titanium alloy Ti-6Al-4V. The elements comprising Ti-6Al-4V and the representative elemental weight percentages of Ti-6A;-4V are titanium (about 90%), aluminum (about 6%), vanadium (about 4%), iron (maximum about 0.25%) and oxygen (maximum about 0.2%). In another embodiment of the present invention, the ultrasonic probe 15 comprises stainless steel. In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel. Those skilled in the art will recognize that the ultrasonic probe can be comprised of many materials known in the art and be within the spirit and scope of the present invention.

In a preferred embodiment of the present invention, the ultrasonic probe 15 has a small diameter. In a preferred embodiment of the present invention, the diameter of the ultrasonic probe 15 gradually decreases from the proximal end 31 to the distal end 24 of the ultrasonic probe 15. In an embodiment of the present invention, the diameter of the distal end 24 of the wire body 63 of the ultrasonic probe 15 is about 0.004 inches. In another embodiment of the present invention, the diameter of the distal end 24 of the wire body 63 of the ultrasonic probe 15 is about 0.015 inches. In other embodiments of the present invention, the diameter of the distal end 24 of the wire body 63 of the ultrasonic probe 15 varies between about 0.003 inches and about 0.025 inches. Those skilled in the art will recognize an ultrasonic probe 15 can have a diameter at the distal end 24 of the wire body 63 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 of the ultrasonic probe 15 smaller than about 0.003 inches, larger than about 0.025 inches, and between about 0.003 inches and about 0.025 inches and be within the spirit and scope of the present invention.

In an embodiment of the present invention, the diameter of the ultrasonic probe 15 is approximately uniform from the proximal end 31 to the distal end 24 of the wire body 63. In another embodiment of the present invention, the diameter of the ultrasonic probe 15 gradually decreases from the proximal end 31 to the distal end 24 of the wire body 63. In an embodiment of the present invention, the ultrasonic probe 15 is a wire. In an embodiment of the present invention, the gradual change of the diameter from the proximal end 31 to the distal end 24 of the wire body 63 occurs over the at least one transition with each transition having an approximately equal length. In another embodiment of the present invention, the gradual change of the diameter from the proximal end 31 to the distal end 24 of the wire body 63 occurs over a plurality of transitions with each transition having a varying length. The transition refers to a section where the diameter varies from a first diameter to a second diameter.

The physical properties (i.e., length, cross sectional shape, dimensions, etc.) and material properties (i.e., yield strength, modulus, etc.) of the ultrasonic probe 15 are selected for operation of the ultrasonic probe 15 in the transverse mode. The length of the ultrasonic probe 15 of the present invention is chosen to be resonant in a transverse mode. In an embodiment of the present invention, the ultrasonic probe 15 is between about 30 centimeters and about 300 centimeters in length. Those skilled in the art will recognize an ultrasonic probe can have a length shorter than about 30 centimeters, a length longer than about 300 centimeters and a length between about 30 centimeters and about 300 centimeters and be within the spirit and scope of the present invention.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer is capable of an acoustic impedance transformation of electrical energy provided by the ultrasonic energy source 99 to mechanical energy. The transducer sets the operating frequency of the ultrasonic medical device 11. The transducer transmits ultrasonic energy received from the ultrasonic energy source 99 to the ultrasonic probe 15. Energy from the ultrasonic energy source 99 is transmitted along the longitudinal axis of the ultrasonic probe 15, causing the ultrasonic probe 15 to vibrate in a transverse mode. The transducer is capable of engaging the ultrasonic probe 15 at the proximal end 31 with sufficient restraint to form an acoustical mass that can propagate the ultrasonic energy provided by the ultrasonic energy source 99.

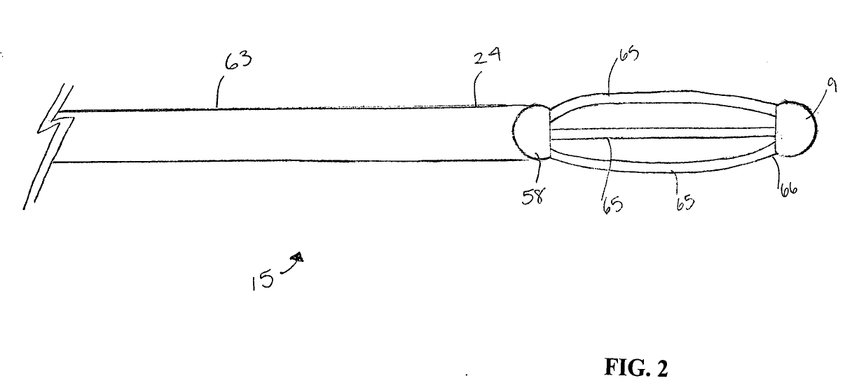

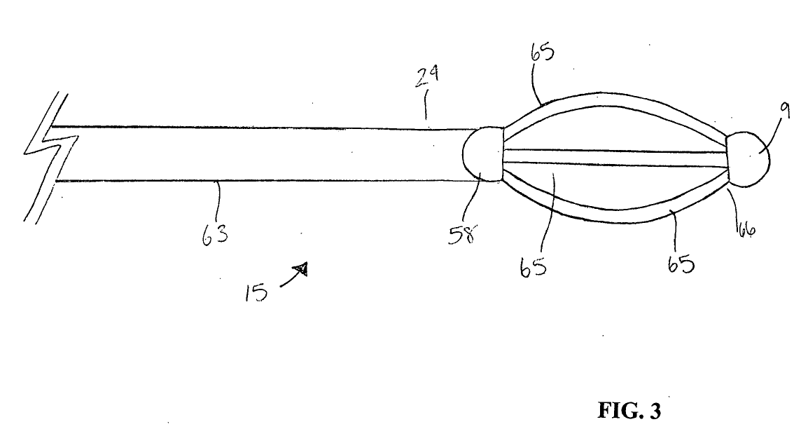

FIG. 2 illustrates a fragmentary side plan of the ultrasonic probe 15 of the present invention with the plurality of tines 65 in a collapsed position. The plurality of tines 65 are connected at the distal end 66. In a preferred embodiment of the present invention, the plurality of tines 65 comprises four tines. In a preferred embodiment of the present invention, the plurality of tines 65 is movable between a collapsed position (FIG. 2) and an expanded position (FIG. 3). A diameter of the plurality of tines 65 in the collapsed position is approximately equal to a diameter of the wire body 63. When the plurality of tines 65 are in a collapsed position, the ultrasonic probe 15 including the plurality of tines 65 is inserted into the sheath 36.

In a preferred embodiment of the present invention, the plurality of tines 65 are engaged to the distal end 24 of the wire body 63. The plurality of tines 65 may engage the distal end 24 of the wire body 63 by welding. In another embodiment of the present invention, the plurality of tines 65 are mechanically fastened to the distal end 24 of the wire body 63. Other mechanisms for engaging the plurality of tines 65 to the distal end 24 of the wire body 63 include, but are not limited to, mechanical fasteners, adhesives, glues, rivets, blind fasteners, mechanical snaps and other fasteners. Those skilled in the art will recognize that other methods of engaging the plurality of tines to the distal end of the wire body are known in the art and would be within the spirit and scope of the present invention.

In a preferred embodiment of the present invention, the plurality of tines 65 are connected together at the distal end 66. Connecting the distal end 66 of the plurality of tines 65 forms a basket-like structure. In another embodiment of the present invention, the plurality of tines 65 are mechanically fastened together at the distal end 66. Other mechanisms for connecting the plurality of tines 65 together at the distal end 66 include, but are not limited to adhesives, glues, rivets, blind fasteners, mechanical snaps and other fasteners. Those skilled in the art will recognize that other methods of connecting the plurality of tines together at the distal end are known in the art and would be within the spirit and scope of the present invention.

FIG. 3 illustrates a fragmentary side plan view of the ultrasonic probe 15 of the present invention with the plurality of tines 65 in an expanded position. The plurality of tines 65 are connected at the distal end 66. When the plurality of tines 65 are in the expanded position, the plurality of tines 65 are outside of the sheath 36. In the expanded position, the diameter of the plurality of tines 65 is larger than a diameter of the wire body 63.

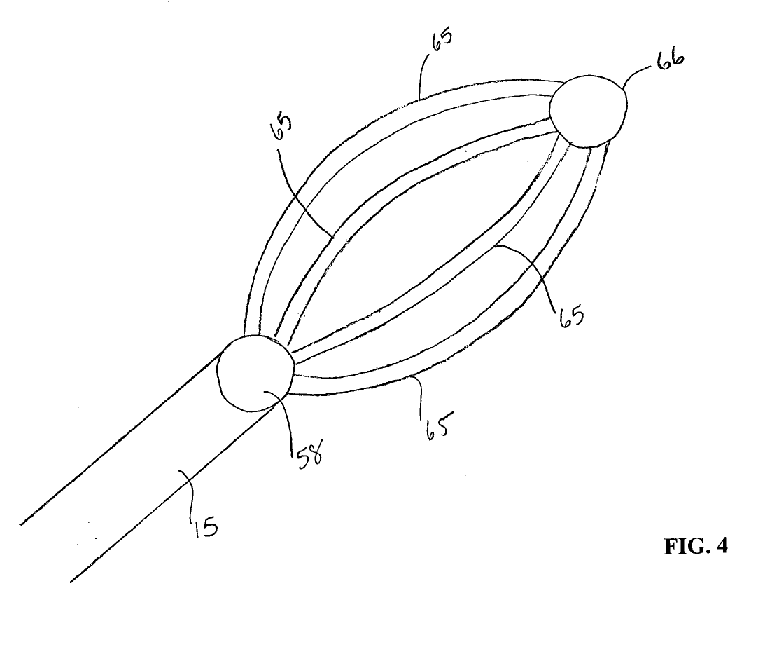

FIG. 4 shows a perspective end view of the plurality of tines 65 of the ultrasonic probe 15. FIG. 4 illustrates the preferred embodiment of the present invention where the plurality of tines 65 comprises four tines 65.

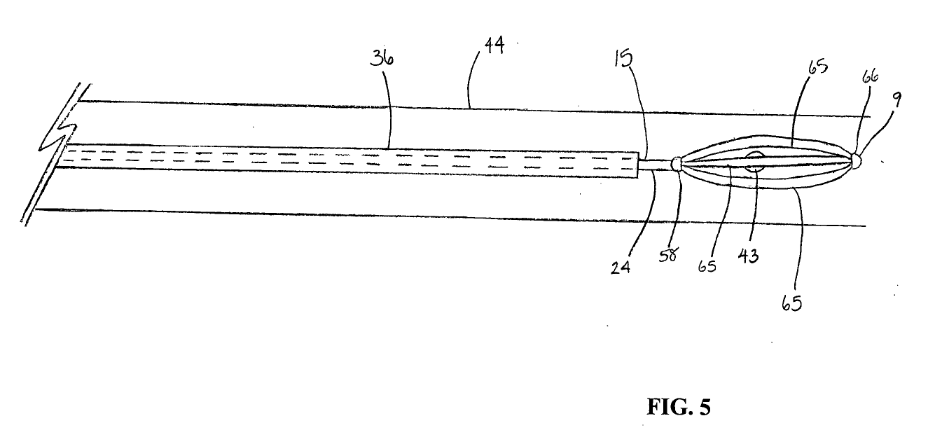

FIG. 5 shows a fragmentary side plan view of the ultrasonic probe 15 of the present invention in an organ 44 of the body with the plurality of tines 65 surrounding a stone 43. The plurality of tines 65 are connected at the distal end 66. With the stone 43 in the organ 44 of the body, a portion of the ultrasonic probe 15 and the sheath 36 are inserted into the body and the ultrasonic probe 15 is moved adjacent to the stone. The ultrasonic probe 15 is moved to place the stone 43 between adjacent tines of the plurality of tines 65 and inside of the plurality of tines 65. The stone 43 is passively constrained within the plurality of tines 65.

A plurality of stones 43 are found within the organs of the body and cause the urolithiasis condition. In one embodiment of the present invention, the stone 43 is a kidney stone. In another embodiment of the present invention, the stone 43 is a gall stone. The stone 43 may also include, but is not limited to, cystine kidney stones, struvite stones, urate stones, calcium oxalate stones, urinary stones, bladder stones, cystine stones, xanthine stones, calcium phosphate stones endemic bladder stones, oxylate stones renal stones, crixivan stones, uric acid stones and uric acid plus calcium stones.

Most commonly, the stones 43 are found within the urinary tract. The urinary tract, also known as the urinary system, consists of the kidneys, ureters, bladder and urethra. The kidneys are two organs shaped like beans located below the ribs toward the middle of the back. The kidneys remove extra water and wastes from the blood, converting it to urine. The kidneys also maintain a stable balance of salts and other substances in the blood and produce hormones that help build strong bones and help form red blood cells. Extending from each kidney is a ureter which carries urine from the kidneys to the bladder, where the urine is temporarily stored. The bladder is a triangle shaped chamber in the lower abdomen whose elastic walls stretch and expand to store urine and flatten together when urine is emptied through the urethra to outside of the body.

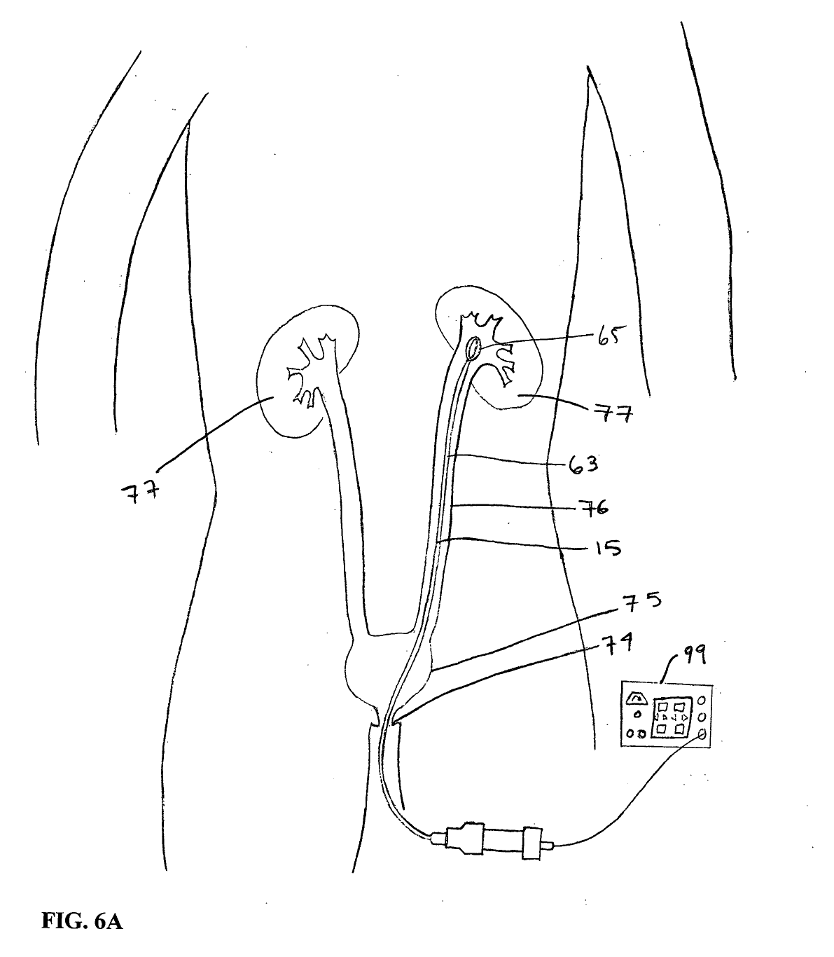

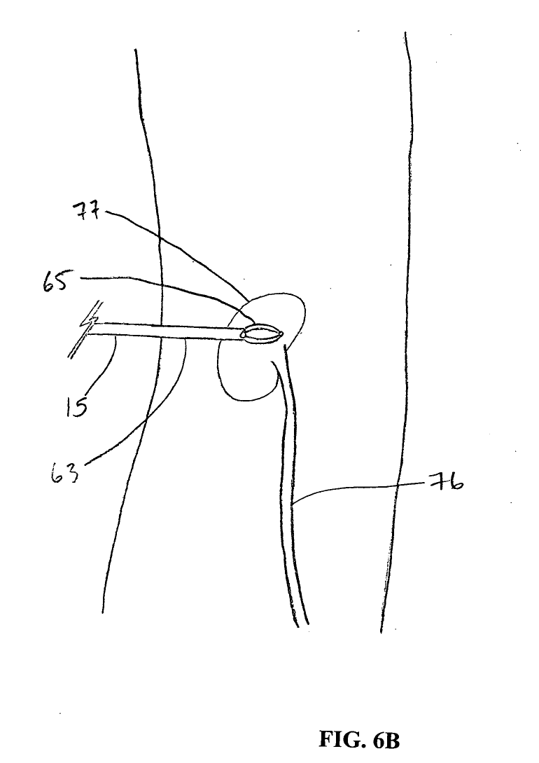

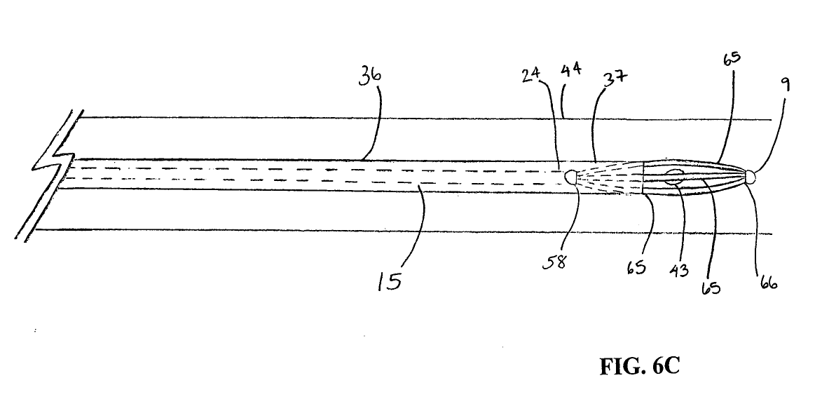

FIG. 6A shows the ultrasonic probe 15 inserted into a kidney 77 of a patient. For the remainder of the patent application, the invention will be described with the stone 43 in the kidney 77. In a preferred embodiment of the present invention, the ultrasonic probe, comprising the wire body 63 and the plurality of tines 65 is inserted through a urethra 74, moved through a bladder 75, up a ureter 76 and into one of the kidneys 77. Aside from the smaller length of the urethra 74, the urinary tract of a male and a female are similar. Once inside the kidney 77, the ultrasonic probe 15 is moved to surround the stone 43 with the plurality of tines 65 to ablate the stone 43.

FIG. 6B shows the ultrasonic probe 15 inserted through the patient's back and into the kidney 77. In this embodiment of the present invention, the ultrasonic probe 15, comprising the wire body 63 and the plurality of tines 65, is inserted through the back and into the kidney 77 to capture the stone 43. In this embodiment of the present invention, a tiny incision is made in the back to create a channel directly into the kidney 77. A device including, but not limited to, a vascular introducer or trocar can be used to create an insertion point in the back to gain access to the kidney 77. A vascular introducer for use with an ultrasonic probe is described in Assignee's co-pending patent application U.S. Ser. No. 10/080,787, and the entirety of this application is hereby incorporated herein by reference.

The ultrasonic medical device 11 of the present invention can be used in conjunction with or after an Endoscopic Retrograde Cholangiopancreatography (ERCP) procedure. ERCP enables a medical professional to diagnose problems in the liver, gallbladder, bile ducts and pancreas. ERCP is used primarily to diagnose and treat conditions of the bile ducts including gallstones, inflammatory strictures, leaks and cancer. In an ERCP procedure, an endoscope is guided through the esophagus, stomach and duodenum until it reaches the spot where the ducts of the biliary tree and pancreas open into the duodenum. A small plastic tube is passed through the endoscope and a dye is injected into the ducts to enable the use of x-rays to determine whether there is a presence of the gall stone or a narrowing of the ducts.

After determining the location of the gall stone, the ultrasonic medical device 11 can be used to remove the gall stone found during the ERCP procedure. In the case where a conventional tool is used to remove the gallstone or the obstruction, it is common to have residual stones in the common bile duct. The removal of the stones from the bile duct is a risky procedure to perform with conventional tools. The ultrasonic medical device 11 provides a safe method of removing the gall stones from the bile duct without compromising the functionality of the bile duct or the health of a patient.

FIG. 6C shows a fragmentary side plan view of the ultrasonic probe 15 of the present invention in the organ 44 of the body with the plurality of tines 65 surrounding the stone 43 and compressed by the sheath 36. The plurality of tines 65 are connected at the distal end 66. A portion of the plurality of tines 65 are compressed by the sheath 36. In a preferred embodiment of the present invention, at least one of the plurality of tines 65 engage an outer surface of the stone 43 when the plurality of tines 65 is in the collapsed position.

The portion of the plurality of tines 65 are compressed by the sheath 36. In one embodiment of the present invention, the sheath 36 is advanced over a portion of the plurality of tines 65 by pushing the proximal end 34 of the sheath 36 toward the plurality of tines 65. In an embodiment of the present invention, the wire body 63 of the ultrasonic probe 15 comprises a plurality of markers. By aligning the end of the proximal end 34 of the sheath 36 with the plurality of markers on the ultrasonic probe 15, the sheath 36 covers a portion of the plurality of tines 65, allowing for the plurality of tines 65 to be compressed while the stone 43 remains distal to the distal end 37 of the sheath 36.

In an embodiment of the present invention, the plurality of markers on the wire body 63 of the ultrasonic probe 15 are comprised of a material of high radiopacity. A material of high radiopacity does not allow the passage of a substantial amount of x-rays or other radiation and allows for the portion of the wire body 63 of the ultrasonic probe 15 at each marker to be detected. The material of high radiopacity allows for the portion of the wire body 63 of the ultrasonic probe 15 at each marker to be visualized and facilitates diagnostic and therapeutic treatments. In another embodiment of the present invention, the plurality of markers are comprised of a material of low radiopacity. An ultrasonic medical device with improved visibility in imaging procedures is described in the Assignee's co-pending patent applications U.S. Ser. No. 10/328,202 and U.S. Ser. No. 10/207,468, and the entirety of these patent applications are hereby incorporated herein by reference. Those skilled in the art will recognize the plurality of markers can be comprised of many materials known in the art and be within the spirit and scope of the present invention.

In another embodiment of the present invention, the ultrasonic probe 15 is pulled back into the distal end 37 of the sheath 36, allowing for the plurality of tines 65 to be compressed, while the stone 43 remains beyond the distal end 37 of the sheath 36. In one embodiment of the present invention, the ultrasonic probe 15 is pulled back until at least one marker on the wire body 63 of the ultrasonic probe 15 aligns with the end of the proximal end 34 of the sheath 36. Those skilled in the art will recognize there are several ways of compressing the plurality of tines known in the art that are within the spirit and scope of the present invention.

With the plurality of tines 65 compressed and engaging the stone 43, the ultrasonic energy source 99 is activated to energize the ultrasonic probe 15. The ultrasonic energy source 99 provides a low power electric signal of about 2 watts to about 15 watts to the transducer that is located within the handle 88. The transducer converts electrical energy provided by the ultrasonic energy source 99 to mechanical energy. The operating frequency of the ultrasonic medical device 11 is set by the transducer and the ultrasonic energy source 99 finds the resonant frequency of the transducer through a Phase Lock Loop. By an appropriately oriented and driven cylindrical array of piezoelectric crystals of the transducer, the horn creates a longitudinal wave along at least a portion of the longitudinal axis of the ultrasonic probe 15. The longitudinal wave is converted to a transverse wave along at least a portion of the longitudinal axis of the ultrasonic probe 15 through a nonlinear dynamic buckling of the ultrasonic probe 15.

As the transverse wave is transmitted along the longitudinal axis of the ultrasonic probe 15, a transverse ultrasonic vibration is created along the longitudinal axis of the ultrasonic probe 15. In a preferred embodiment of the present invention, the ultrasonic probe 15 including the wire body 63 and the plurality of tines 65 vibrate in a direction transverse (not parallel) to the axial direction. The transverse ultrasonic vibration propagates along the ultrasonic probe 15 including the plurality of tines 65. The transverse mode of vibration of the ultrasonic probe 15 differs from an axial (or longitudinal) mode of vibration disclosed in the prior art. The transverse ultrasonic vibrations along the longitudinal axis of the ultrasonic probe 15 create a plurality of energetic transverse nodes and a plurality of energetic transverse anti-nodes along a portion of the longitudinal axis of the ultrasonic probe 15.

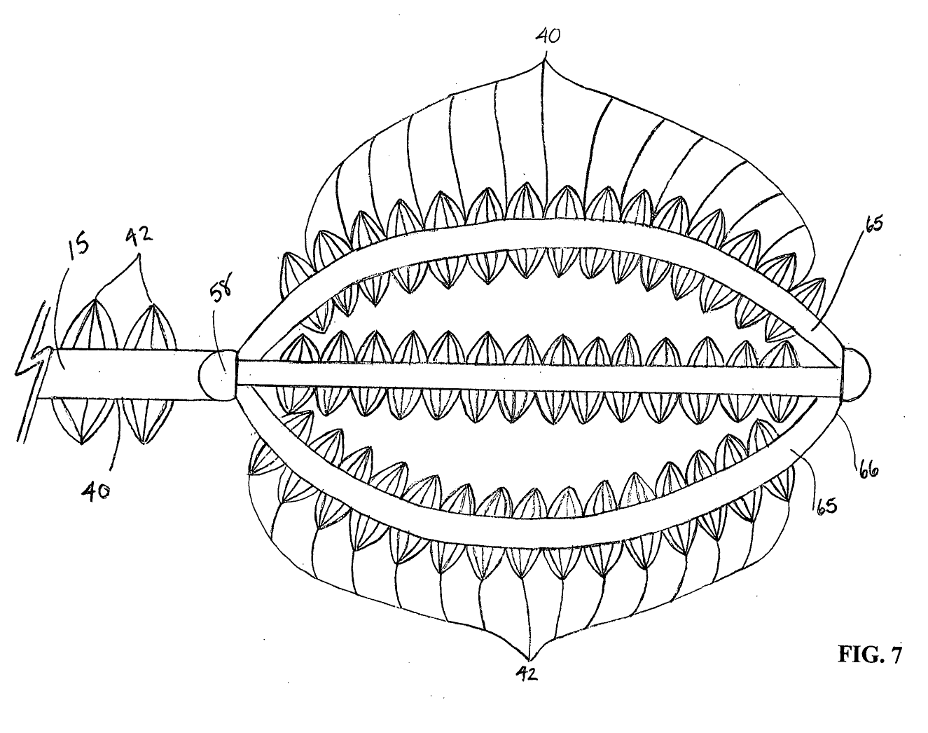

FIG. 7 shows a fragmentary side plan view of the ultrasonic probe 15 of the present invention showing a plurality of transverse nodes 40 and a plurality of transverse anti-nodes 42 along the plurality of tines 65. The transverse nodes 40 are areas of minimum energy and minimum vibration. The transverse anti-nodes 42, or areas of maximum energy and maximum vibration, also occur at repeating intervals along the portion of the ultrasonic probe 15. The number of transverse nodes 40 and transverse anti-nodes 42, and the spacing of the transverse nodes 40 and transverse anti-nodes 42 of the ultrasonic probe 15 depend on the frequency of energy produced by the ultrasonic energy source 99. The separation of the transverse nodes 40 and the transverse anti-nodes 42 is a function of the frequency, and can be affected by tuning the ultrasonic probe 15. In a properly tuned ultrasonic probe 15, the transverse anti-nodes 42 will be found at a position approximately one-half of the distance between the transverse nodes 40 located adjacent to each side of the transverse anti-nodes 42.

With the plurality of tines 65 engaging the stone 43 the transverse wave is transmitted along the ultrasonic probe 15 to the plurality of tines 65, and the interaction of the surface of the ultrasonic probe 15 including the plurality of tines 65 with a medium surrounding the ultrasonic probe 15, including the plurality of tines 65, creates an acoustic wave in the surrounding medium. As the transverse wave is transmitted along the ultrasonic probe 15, the ultrasonic probe 15 including the plurality of tines 65 vibrates transversely. The transverse motion of the ultrasonic probe 15 produces cavitation in the medium surrounding the ultrasonic probe 15 to ablate the stone 43. Through a process of cavitation, the transverse wave generates acoustic energy in the surrounding fluid. Cavitation is a process in which small voids are formed in a surrounding medium through rapid motion of the ultrasonic probe 15 and the voids are subsequently forced to compress. The compression of the voids creates a wave of acoustic energy which acts to dissolve the stone 43, while having no damaging effects on healthy tissue. The acoustic energy from the pressure wave is transmitted to the stone 43. The plurality of tines 65 of the ultrasonic probe 15 increases a surface area of the ultrasonic probe 15 in communication with the stone 43 and the plurality of tines 65 focus a stone destroying effect of the ultrasonic probe 15 to break up the stone 43.

In a preferred embodiment of the present invention, the stone 43 is resolved into a particulate comparable in size to red blood cells (about 5 microns in diameter). The plurality of tines 65 expand a treatment area of the ultrasonic probe 15 and provide a large active area of the ultrasonic probe 15 to communicate with the stone 43. By surrounding at least a portion of the outer surface of the stone 43 with the plurality of tines 65, the surface area of the ultrasonic probe in communication with the stone 43 is increased and the acoustic energy is imparted on the outer surface of the stone 43. The acoustic energy penetrates into the stone 43 and the stone 43 is broken into a particulate that is easily discharged from the body through conventional ways or simply dissolves into the blood stream. A conventional way of discharging the particulate from the body includes transferring the particulate through the blood stream to the kidney where the particulate is excreted as bodily waste.

The ultrasonic energy source 99 produces a transverse ultrasonic vibration along the wire body 63 and the plurality of tines 65. The ultrasonic probe 15 can support the transverse ultrasonic vibration along the wire body 63 and the plurality of tines 65. The transverse mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates in a direction transverse (not parallel) to the axial direction.

Transversely vibrating ultrasonic probes for biological material ablation are described in the Assignee's U.S. Pat. No. 6,551,337; U.S. Pat. No. 6,652,547; and U.S. Pat. No. 6,660,013 and Assignee's co-pending patent application U.S. Ser. No. 09/917,471, which further describe the design parameters for such an ultrasonic probe and its use in ultrasonic devices for ablation, and the entirety of these patents and patent applications are hereby incorporated herein by reference.

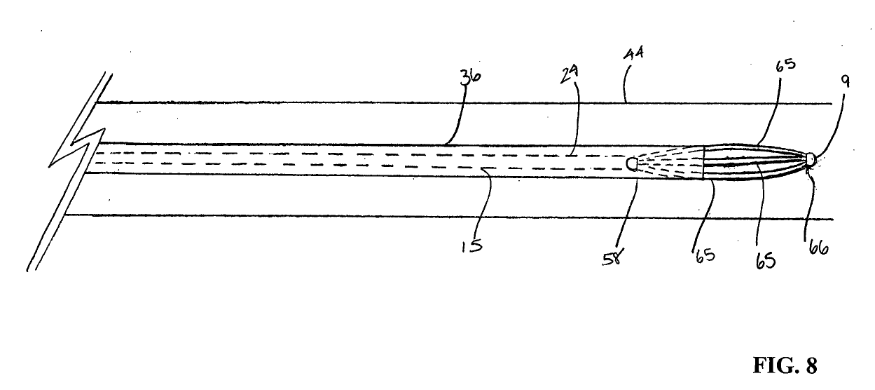

FIG. 8 shows a fragmentary side plan view of the ultrasonic probe 15 of the present invention in the organ of the body after the stone 43 has been ablated. The sheath 36 is moved over and compresses a portion of the plurality of tines 65. The distal end 66 of the plurality of tines 65 are connected.

In another embodiment of the present invention, the acoustic energy is imparted onto the outer surface of the stone 43 and the acoustic energy penetrates into the stone 43 as the stone 43 is broken into a plurality of fragments that are not easily discharged from the body. A diameter of the plurality of fragments of the stone 43 is smaller than the inner diameter of the sheath 36. With the plurality of fragments of the stone 43 residing within the plurality of tines 65, the ultrasonic probe 15 is pulled back toward the outside of the body and the entire plurality of tines 65 surrounding the fragments of the stone 43 is pulled within the sheath 36. The ultrasonic medical device 11 is pulled out of the organ 44 of the body, allowing for the plurality of fragments of stone 43 to be removed from the organ 44 of the body.

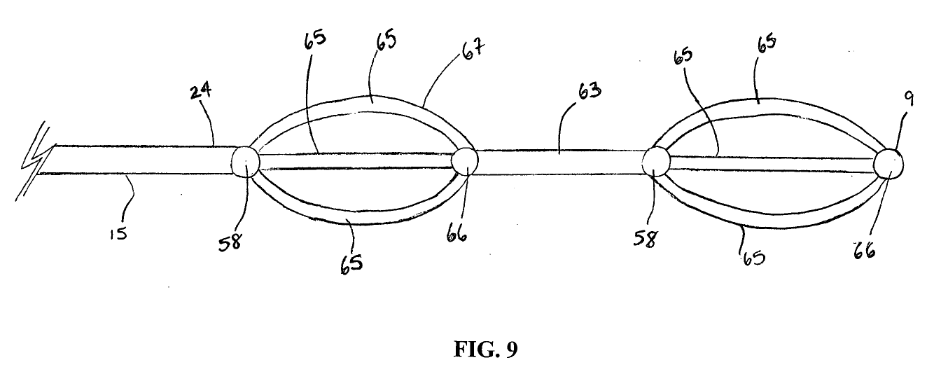

FIG. 9 shows an alternative embodiment of the present invention with the ultrasonic probe 15 comprising a plurality of wire body segments 63 and a plurality of sections comprising a plurality of tines 65. The plurality of tines 65 are connected at the distal end 66. The ultrasonic probe 15 shown in FIG. 9 allows for the removal of one or more stones 43 in the organ 44 of the body simultaneously. In the embodiment of the present invention shown in FIG. 9, a plurality of tines are located at the distal end of the ultrasonic probe and a second plurality of tines 65 are located along the longitudinal axis of the ultrasonic probe 15. The ultrasonic probe 15 can capture a first stone 43 with the plurality of tines 65 located at the distal end of the ultrasonic probe 15 and a second stone 43 with the second plurality of tines 65 located along the longitudinal axis of the ultrasonic probe 15.

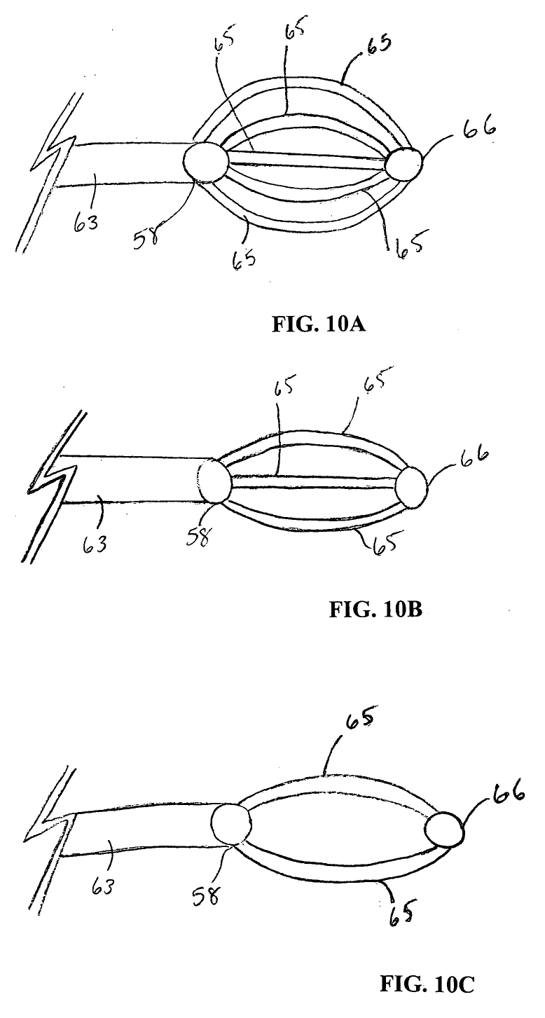

In a preferred embodiment of the present invention, the plurality of tines 65 of the ultrasonic probe 15 comprises four tines. In another embodiment of the present invention shown in FIG. 10A, the plurality of tines 65 of the ultrasonic probe 15 comprises five tines. The embodiment shown in FIG. 10A is effective for ablating large stones 43, where the stone destroying effects of the ultrasonic probe 15 are increased as the surface area of the ultrasonic probe 15 in communication with the large stone 43 is increased by more tines 65 contacting the stone 43. In another embodiment of the present invention shown in FIG. 10B, the plurality of tines 65 of the ultrasonic probe 15 comprises three tines. The embodiment shown in FIG. 10B is effective for ablating small or medium sized stones 43. In another embodiment of the present invention shown in FIG. 10C, the plurality of tines 65 of the ultrasonic probe 15 comprises two tines. The embodiment shown in FIG. 10C is effective for ablating small stones 43. Those skilled in the art will recognize the plurality of tines can be comprised of any number of tines and be within the spirit and scope of the present invention.

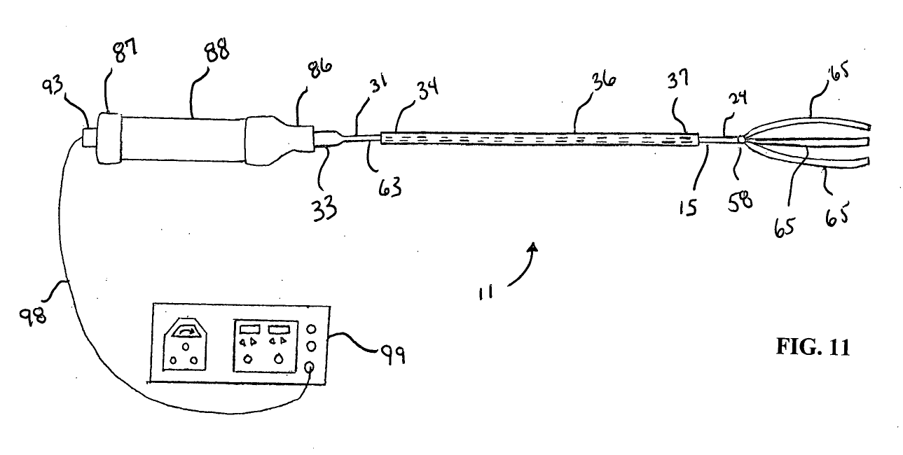

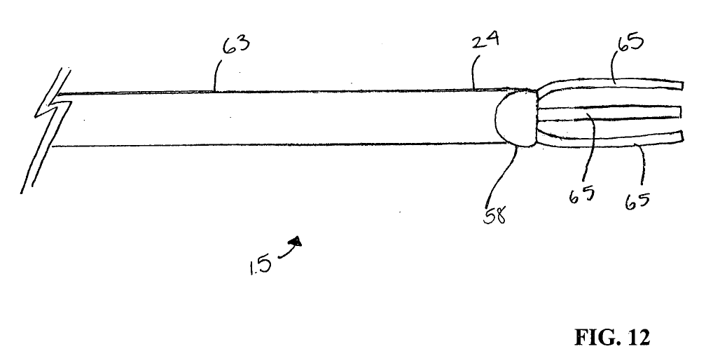

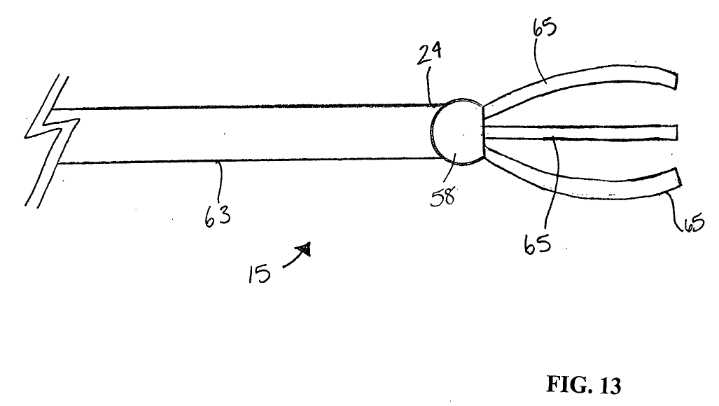

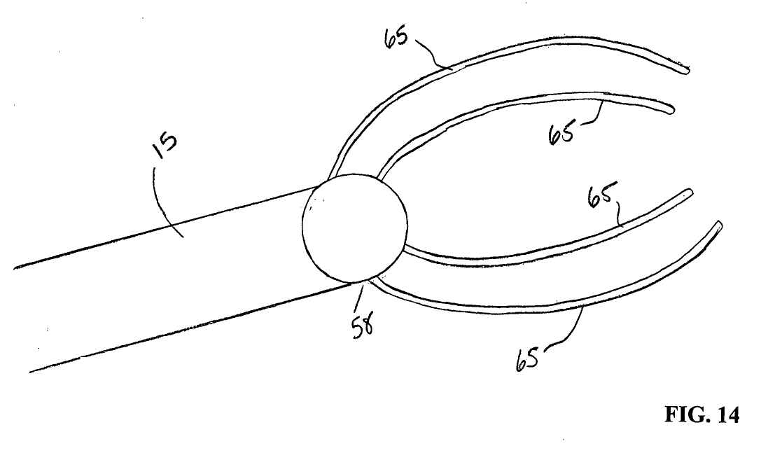

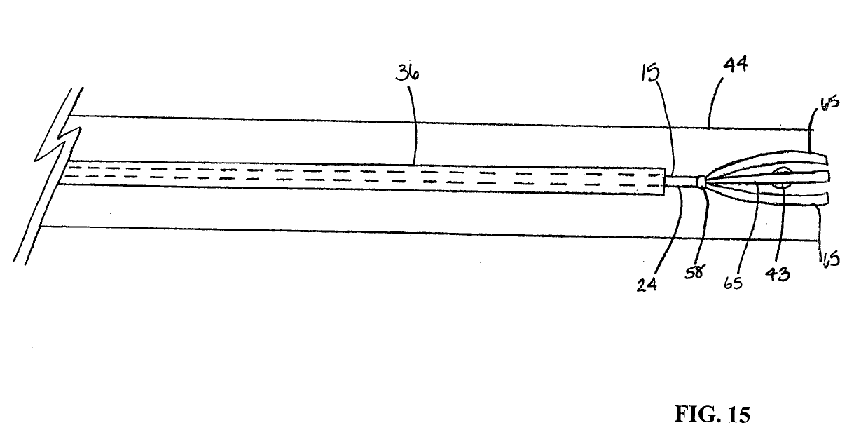

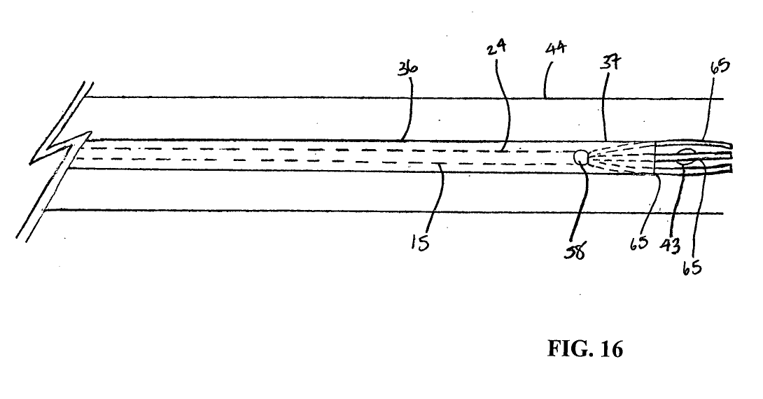

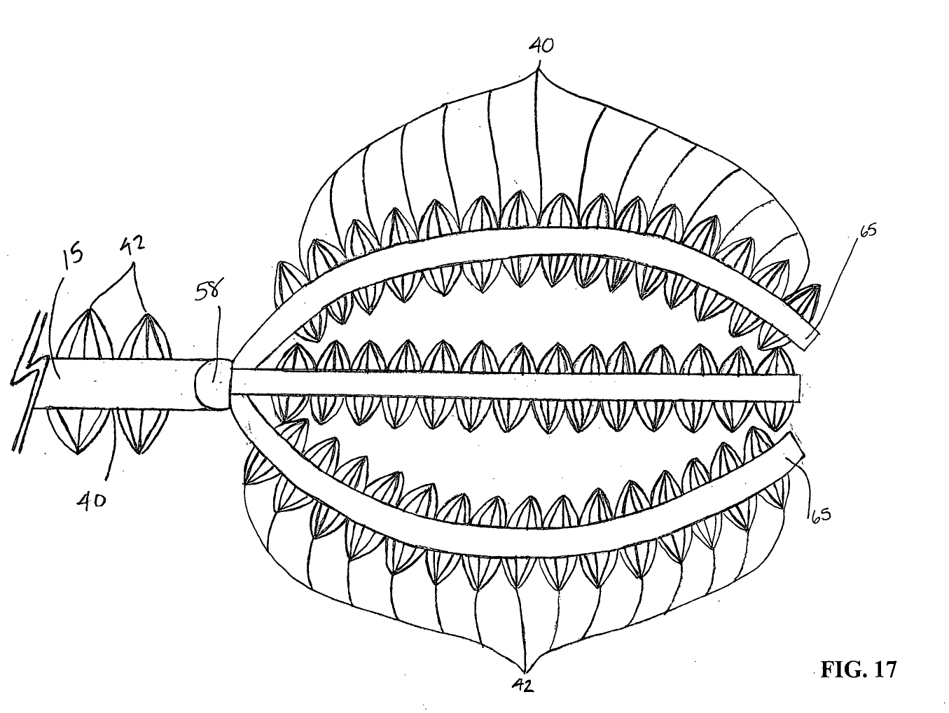

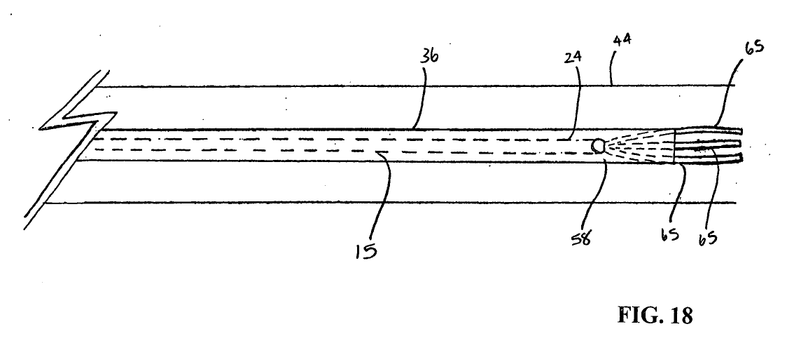

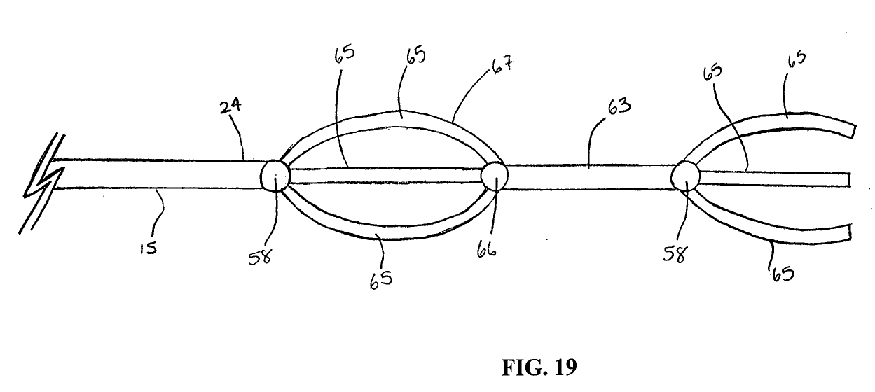

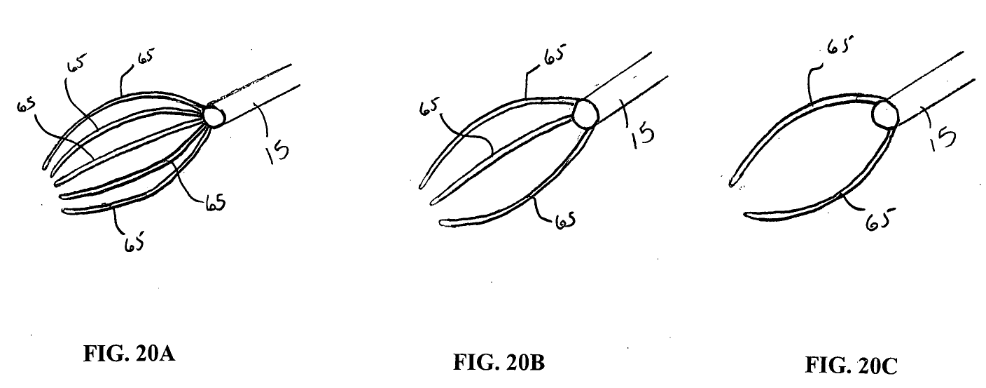

FIGS. 11-20C illustrate the alternative embodiment of the present invention where the plurality of tines 65 are not connected at the distal end 66. FIGS. 11-20C are similar to FIGS. 1-10C except the plurality of tines 65 are not connected at the distal end 66. Because the plurality of tines 65 are not connected at the distal end 66, each tine of the plurality of tines 65 can move independent of the other tines. This embodiment is effective for reaching and ablating stones in difficult to reach locations. Discussion of the structure and functionality of the ultrasonic medical device 11 for FIGS. 1-10C applies to the alternative embodiment shown in FIGS. 11-20C.

In the alternative embodiment shown in FIGS. 11-20C where the plurality of tines 65 are not connected at the distal end 66, the stone 43 is surrounded within the plurality of tines 65 by moving the distal end 66 toward the stone 43. The stone 43 enters the distal end 66 of the plurality of tines 65 and the plurality of tines 65 surround the stone. The embodiment of the present invention shown in FIGS. 1 1-20C is useful for large stones 43 found in various organs 44 of the body, where the capture of the stone 43 within the plurality of tines 65 is easier with the plurality of tines 65 not connected at the distal end 66.

The present invention provides a method of ablating a stone 43 in an organ 44 of the body in a minimally invasive manner. The ultrasonic probe 15, comprising the wire body 63 and the plurality of tines 65 extending from a distal end 24 of the wire body 63, is inserted into the sheath 36, moving the plurality of tines 65 into the collapsed position. The plurality of tines of the ultrasonic probe 15 is moved from the collapsed position into the expanded position by advancing the plurality of tines beyond the distal end 37 of the sheath 36. The ultrasonic probe 15 is moved until the plurality of tines 65 surround at least a portion of the outer surface of the stone 43. A portion of the plurality of tines 65 are compressed by the sheath 36 to engage the stone 43 with the plurality of tines 65. The ultrasonic energy source 99 is activated to provide the ultrasonic energy to the ultrasonic probe 15 to ablate the stone 43.

The plurality of tines 65 and the wire body 63 are vibrated in a direction transverse to the axial direction. The transverse ultrasonic vibrations along the plurality of tines 65 and the wire body 63 create a plurality of energetic transverse nodes and a plurality of energetic transverse anti-nodes along a portion of the longitudinal axis of the ultrasonic probe 15. Through a process of cavitation, the transverse wave generates acoustic energy in the surrounding fluid. Small voids are formed and forced to compress through rapid motion of the ultrasonic probe 15, creating a wave of acoustic energy which acts to ablate the stone 43.

The present invention also provides a method of reducing a size of an at least one stone in an organ 44 of the body. The ultrasonic probe 15 is inserted into a biocompatible material member, the biocompatible material member comprising a wire body 63 with a plurality of tines 65 engaging the wire body 63. The plurality of tines 65 of the ultrasonic probe 15 is moved from the collapsed position to the expanded position by advancing the plurality of tines 65 beyond the distal end 37 of the biocompatible material member. The ultrasonic probe 15 is moved until the plurality of tines 65 surround at least a portion of the outer surface of the stone 43. The ultrasonic energy source 43 is energized to produce a transverse ultrasonic vibration along the ultrasonic probe 15.

In an alternative embodiment of the present invention, the ultrasonic probe 15 vibrates in a torsional mode. In the torsional mode of vibration, a portion of the longitudinal axis of the ultrasonic probe 15 comprises a radially asymmetric cross section and the length of the ultrasonic probe 15 is chosen to be resonant in the torsional mode. In the torsional mode of vibration, a transducer transmits ultrasonic energy received from the ultrasonic energy source 99 to the ultrasonic probe 15, causing the ultrasonic probe 15 to vibrate torsionally. The ultrasonic energy source 99 produces the electrical energy that is used to produce a torsional vibration along the longitudinal axis of the ultrasonic probe 15. The torsional vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of a plurality of torsional nodes and a section distal to each of the plurality of torsional nodes are vibrated out of phase, with the proximal section vibrated in a clockwise direction and the distal section vibrated in a counterclockwise direction, or vice versa. The torsional vibration results in an ultrasonic energy transfer to the biological material with minimal loss of ultrasonic energy that could limit the effectiveness of the ultrasonic medical device 11. The torsional vibration produces a rotation and a counterrotation along the longitudinal axis of the ultrasonic probe 15 that creates the plurality of torsional nodes and a plurality of torsional anti-nodes along a portion of the longitudinal axis of the ultrasonic probe 15 resulting in cavitation along the portion of the longitudinal axis of the ultrasonic probe 15 comprising the radially asymmetric cross section in a medium surrounding the ultrasonic probe 15 that ablates the biological material. An apparatus and method for an ultrasonic medical device operating in a torsional mode is described in Assignee's co-pending patent application U.S. Ser. No. 10/774,985, and the entirety of this application is hereby incorporated herein by reference.

In another embodiment of the present invention, the ultrasonic probe 15 vibrates in a torsional mode and a transverse mode. A transducer transmits ultrasonic energy from the ultrasonic energy source 99 to the ultrasonic probe 15, creating a torsional vibration of the ultrasonic probe 15. The torsional vibration induces a transverse vibration along an active area of the ultrasonic probe 15, creating a plurality of nodes and a plurality of anti-nodes along the active area that result in cavitation in a medium surrounding the ultrasonic probe 15. The active area of the ultrasonic probe 15 undergoes both the torsional vibration and the transverse vibration.

Depending upon physical properties (i.e., length, diameter, etc.) and material properties (i.e., yield strength, modulus, etc.) of the ultrasonic probe 15, the transverse vibration is excited by the torsional vibration. Coupling of the torsional mode of vibration and the transverse mode of vibration is possible because of common shear components for the elastic forces. The transverse vibration is induced when the frequency of the transducer is close to a transverse resonant frequency of the ultrasonic probe 15. The combination of the torsional mode of vibration and the transverse mode of vibration is possible because for each torsional mode of vibration, there are many close transverse modes of vibration. By applying tension on the ultrasonic probe 15, for example by bending the ultrasonic probe 15, the transverse vibration is tuned into coincidence with the torsional vibration. The bending causes a shift in frequency due to changes in tension. In the torsional mode of vibration and the transverse mode of vibration, the active area of the ultrasonic probe 15 is vibrated in a direction not parallel to the longitudinal axis of the ultrasonic probe 15 while equally spaced points along the longitudinal axis of the ultrasonic probe 15 in a proximal section vibrate back and forth in a short arc about the longitudinal axis of the ultrasonic probe 15. An apparatus and method for an ultrasonic medical device operating in a transverse mode and a torsional mode is described in Assignee's co-pending patent application U.S. Ser. No. 10/774,898, and the entirety of this application is hereby incorporated herein by reference.

In another embodiment of the present invention, the ultrasonic probe 15 including the plurality of tines vibrates in a longitudinal direction. In another embodiment of the present invention, the ultrasonic probe 15 including the plurality of tines 65 vibrates in a longitudinal direction and a transverse direction. Longitudinal and transverse motion of the ultrasonic probe 15 and the plurality of tines 65 work together to more effectively ablate the stone 43. Those skilled in the art will recognize the ultrasonic probe can vibrate in different directions and be within the spirit and scope of the present invention.

The present invention provides an apparatus and a method for using an ultrasonic medical device to treat urolithiasis. A stone 43 in an organ 44 of the body is surrounded by a plurality of tines 65 of the ultrasonic probe 15. A wave of acoustic energy is formed around the plurality of tines 65 and the wire body, ablating the stone 43. The present invention provides an apparatus and a method for treating urolithiasis that is simple, effective, safe, time efficient and reliable.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An ultrasonic medical device for removing a stone in an organ of a body comprising:
a wire body having a proximal end, a distal end and a longitudinal axis therebetween;
a plurality of tines extending from the distal end of the wire body for engaging the stone; and
a sheath capable of surrounding the wire body and the plurality of tines, wherein a transverse ultrasonic vibration propagates along the wire body and the plurality of tines.

2. The ultrasonic medical device of claim 1 wherein the plurality of tines form a basket-like structure.

3. The ultrasonic medical device of claim 1 wherein the wire body and the plurality of tines form an ultrasonic probe of the ultrasonic medical device.

4. The ultrasonic medical device of claim 1 wherein a distal end of the plurality of tines are connected.

5. The ultrasonic medical device of claim 1 wherein a distal end of the plurality of tines are not connected.

6. The ultrasonic medical device of claim 1 wherein a proximal end of the plurality of tines is welded to the wire body.

7. The ultrasonic medical device of claim 1 wherein a proximal end of the plurality of tines is mechanically fastened to the wire body.

8. The ultrasonic medical device of claim 1 wherein the plurality of tines are movable between a collapsed position and an expanded position.

9. The ultrasonic medical device of claim 1 wherein a diameter of the plurality of tines in a collapsed position is approximately equal to a diameter of the wire body.

10. The ultrasonic medical device of claim 1 wherein a diameter of the plurality of tines in an expanded position after removing the plurality of tines from within the sheath is larger than a diameter of the wire body.

11. The ultrasonic medical device of claim 1 wherein the plurality of tines surround at least a portion of an outer surface of the stone.

12. The ultrasonic medical device of claim 1 wherein the plurality of tines focus a stone destroying effect of the ultrasonic medical device.

13. The ultrasonic medical device of claim 1 wherein the plurality of tines engage the stone.

14. The ultrasonic medical device of claim 1 further comprising a plurality of markers on the wire body.

15. The ultrasonic medical device of claim 3 further comprising an ultrasonic energy source engaged to the ultrasonic probe that supplies an ultrasonic energy to the ultrasonic probe.

16. The ultrasonic medical device of claim 3 wherein the transverse ultrasonic vibration provides a plurality of transverse nodes and a plurality of transverse anti-nodes along at least a portion of the ultrasonic probe including the plurality of tines.

17. An ultrasonic probe for ablation of at least one stone in an organ of a body comprising:
a wire body having a proximal end, a distal end and a longitudinal axis therebetween; and
a plurality of tines engaging the wire body,
wherein an ultrasonic energy source engaged to the ultrasonic probe supplies an ultrasonic energy to the ultrasonic probe, producing a transverse ultrasonic vibration along at least a portion of the ultrasonic probe to ablate the stone.

18. The ultrasonic probe of claim 17 wherein the plurality of tines extend from a distal end of the wire body.

19. The ultrasonic probe of claim 17 wherein the plurality of tines engage the wire body between a proximal end and a distal end of the wire body.

20. The ultrasonic probe of claim 17 further comprising a second plurality of tines engaging the wire body of the ultrasonic probe.

21. The ultrasonic probe of claim 20 wherein at least one of the plurality of tines is located between the distal end and the proximal end of the wire body.

22. The ultrasonic probe of claim 17 wherein the ultrasonic probe is disposable.

23. The ultrasonic probe of claim 17 wherein the ultrasonic probe is for a single use on a single patient.

24. The ultrasonic probe of claim 17 wherein the plurality of tines are movable between a collapsed position and an expanded position.

25. The ultrasonic probe of claim 17 wherein a diameter of the plurality of tines in a collapsed position is approximately equal to a diameter of the wire body.

26. The ultrasonic probe of claim 17 wherein a diameter of the plurality of tines in an expanded position is larger than a diameter of the wire body.

27. The ultrasonic probe of claim 17 wherein the plurality of tines focus a stone destroying effect of the ultrasonic probe.

28. The ultrasonic probe of claim 17 wherein the plurality of tines engage at least one stone.

29. The ultrasonic probe of claim 17 wherein a distal end of the plurality of tines are connected.

30. The ultrasonic probe of claim 17 wherein a distal end of the plurality of tines are not connected.

31. The ultrasonic probe of claim 17 wherein the plurality of tines surround at least a portion of an outer surface of at least one stone.

32. The ultrasonic probe of claim 17 wherein the transverse ultrasonic vibration produces a plurality of transverse nodes and a plurality of transverse anti-nodes along at least a portion of the ultrasonic probe including the plurality of tines.

33. A method of ablating a stone in an organ of a body comprising:
inserting an ultrasonic probe into a sheath, the ultrasonic probe having a wire body and a plurality of tines extending from a distal end of the wire body;
moving the plurality of tines from a collapsed position to an expanded position by advancing the plurality of tines beyond a distal end of the sheath;
moving the ultrasonic probe until the plurality of tines surround at least a portion of an outer surface of the stone;
compressing a portion of the plurality of tines to engage the stone; and
activating an ultrasonic energy source to provide an ultrasonic energy to the ultrasonic probe to ablate the stone.

34. The method of claim 33 further comprising pushing the ultrasonic probe through the sheath to advance the plurality of tines beyond the distal end of the sheath.

35. The method of claim 33 further comprising pulling back on the sheath to advance the plurality of tines beyond the distal end of the sheath.

36. The method of claim 33 further comprising compressing the plurality of tines by pulling the portion of the plurality of tines back into the sheath.

37. The method of claim 33 further comprising compressing the plurality of tines by moving the sheath over the portion of the plurality of tines.

38. The method of claim 33 further comprising producing a transverse ultrasonic vibration along the ultrasonic probe by the ultrasonic energy source.

39. The method of claim 38 wherein the transverse ultrasonic vibration provides a plurality of transverse nodes and a plurality of transverse anti-nodes along at least a portion of the ultrasonic probe.

40. The method of claim 33 further comprising moving the ultrasonic probe back and forth to surround the stone within the plurality of tines.

41. The method of claim 33 further comprising sweeping the ultrasonic probe to surround the stone within the plurality of tines.

42. The method of claim 33 further comprising rotating the ultrasonic probe to surround the stone within the plurality of tines.

43. The method of claim 33 further comprising twisting the ultrasonic probe to surround the stone within the plurality of tines.

44. The method of claim 33 wherein a distal end of the plurality of tines are connected.

45. The method of claim 33 wherein a distal end of the plurality of tines are not connected.

46. The method of claim 33 wherein a diameter of the plurality of tines in the expanded position after removing the plurality of tines from within the sheath is larger than a diameter of the wire body.

47. The method of claim 33 wherein a diameter of the plurality of tines in the collapsed position is approximately equal to a diameter of the wire body.

48. The method of claim 33 further comprising focusing a stone destroying effect of the ultrasonic probe through the plurality of tines.

49. A method of reducing a size of a stone in an organ of a body comprising:
inserting an ultrasonic probe into a biocompatible material member, the ultrasonic probe comprising a wire body with a plurality of tines engaging the wire body;
moving the plurality of tines from a collapsed position to an expanded position by advancing the plurality of tines beyond a distal end of the biocompatible material member;
moving the ultrasonic probe until the plurality of tines surround at least a portion of an outer surface of the stone; and
activating an ultrasonic energy source to produce a transverse ultrasonic vibration along the ultrasonic probe to reduce the size of the stone.

50. The method of claim 49 wherein the biocompatible material member is selected from the group consisting of a catheter, a balloon, and a sheath.

51. The method of claim 49 further comprising compressing the plurality of tines to engage the stone.

52. The method of claim 49 further comprising reducing the stone to a size that can be discharged from the body in a conventional way.

53. The method of claim 49 further comprising reducing the stone to a size smaller than an inner diameter of the biocompatible material member and pulling the stone through the biocompatible material member to remove at least one stone from the body.

54. The method of claim 49 further comprising producing the transverse ultrasonic vibration to provide a plurality of transverse nodes and a plurality of transverse anti-nodes along at least a portion of the ultrasonic probe.

55. The method of claim 49 further comprising focusing a stone destroying effect of the ultrasonic probe through the plurality of tines.

56. The method of claim 49 further comprising providing the transverse ultrasonic vibration along the wire body and the plurality of tines.
